(19) United States
(12) Patent Application Publication
(10) Pub. No.: US 2013/0289370 A1
(43) Pub. Date: Oct. 31, 2013

(54) METHOD AND DEVICE FOR DETECTING A BLOOD GLUCOSE LEVEL USING A ELECTROMAGNETIC WAVE

(71) Applicants: Chi-Kuang Sun, Taipei (TW); Yuan-Fu Tsai, Taipei (TW); Hua Chen, Taipei (TW)

(72) Inventors: Chi-Kuang Sun, Taipei (TW); Yuan-Fu Tsai, Taipei (TW); Hua Chen, Taipei (TW)

(73) Assignee: NATIONAL TAIWAN UNIVERSITY, Taipei (TW)

(21) Appl. No.: 13/684,408

(22) Filed: Nov. 23, 2012

(30) Foreign Application Priority Data
Apr. 25, 2012 (TW) 101114805

(51) Int. Cl. A61B 5/145; A61B 5/00; A61B 5/1455

(52) U.S. Cl. CPC A61B 5/14532; A61B 5/1455; A61B 5/7246
USPC 600/316

(57) ABSTRACT

The present invention provides a method for detecting a blood glucose level of a subject using an electromagnetic wave. Because a different blood glucose level is accompanied by a different electromagnetic absorption constant, the present invention compares a detected blood glucose electromagnetic absorption constant of the subject with data in a blood glucose electromagnetic absorption constant database so as to obtain a blood glucose concentration of the subject. The present invention also provides a device for detecting a blood glucose level of the subject using the electromagnetic wave.

### CROSS-REFERENCE TO RELATED APPLICATION

This application claims the benefit of Taiwan Patent Application No. 101114805, filed on Apr. 25, 2012, in Taiwan Intellectual Property Office, the disclosure of which is incorporated herein by reference.

### FIELD OF THE INVENTION

This invention relates to a method and device for noninvasively detecting a blood glucose level.

### DESCRIPTION OF PRIOR ART

It is desired to provide a novel, noninvasive method to improve the current, inconvenient blood glucose detecting method, having great potential to replace the current invasive detecting method and applying to diagnose diabetes in clinical practice.

Diabetes mellitus (DM) refers to a group of common metabolic disorders that share the phenotype of hyperglycemia. Depending on the etiology of the DM, factors contributing to hyperglycemia include reduced insulin secretion, decreased glucose utilization, and increased glucose production. The metabolic dysregulation associated with DM causes secondary pathophysiologic changes in multiple organ systems that impose a tremendous burden on the individual with diabetes and on the health care system. In the United States, DM is the leading cause of end-stage renal disease (ESRD), non-traumatic lower extremity amputations, and adult blindness. It also predisposes to cardiovascular diseases. In 2005, according to the World Health Organization, at least 346 million people worldwide suffer from diabetes. Its incidence is increasing rapidly, and it is estimated that by 2030, this number will almost double. Diabetes mellitus occurs throughout the world, but is more common (especially type 2) in the more developed countries. The diagnosis of diabetes includes an oral glucose tolerance test, fasting plasma glucose test, and random plasma glucose test.

Invasive Techniques

The most common invasive blood glucose detecting devices are glucose strips along with hand-held glucose meters and implantable sensor, the former one records glucose levels in blood drawn intravenously by pricking blood on skin via needles or micro-needles (such as: Exac-Tech RSG, ABBOTT LABS; Amira AtLast, AMIRA MEDICAL; and Fast Take, LIFE SCAN), and the later one measures glucose concentration subcutaneously by analyzing the interstitial water or tissue via microdialysis, optical sensing such as fluorescence, or ultrasound transdermal glucose monitoring (such as: Minimed Paradigm, MEDTRONIC and DexCom systems, DEXCOM) These approaches are short on health risks due to the sensor implantation, infection, patient inconvenience, and measurement delay that users will not be willing to use and will cause the risk of infection. Furthermore, the disposable test strip or the probes will cause an extra payment.

Non-Invasive Techniques

Recently, varied techniques were employed on non-invasive blood glucose detecting device, such as reverse iontophoresis (ex. Gluco Watch Automatic Glucose Biographer and Auto Sensors, CYGNUS INC.), bioimpedance measurement (Pendra, PENDRAGON MEDICAL LTD.), metabolic heat conformation, photoacoustic spectroscopy (US 20120172686, Jul. 5 2012), and dielectric spectroscopy. Utilized spectroscopic techniques include: Raman (US 20050090750, Apr. 28 2005), fluorescence, as well as techniques using light from ultraviolet through the ultraviolet (200˜400 nm), visible (400˜700 nm), near-IR (700˜2500 nm or 14286˜4000 cm-1) (U.S. Pat. No. 7,787,924, Aug. 31 2010; and US 20050010090, Jan. 13 2005), infrared (2500˜14285 nm or 4000˜700 cm-1), and microwave (>1 cm) (S. K. Vashist, Analytica Chimica Acta. 2012).

The above techniques still have some limitations or disadvantages for measurements. Reverse iontophoresis technique requires a certain minimum duration and would be strongly interfered with sweating. The skin irritation observed in human subjects is the major drawback of the reverse iontophoresis technique. The measurement of Near-infrared spectroscopy (NIR) technique was sensitive to and easily being be interfered by the physicochemical parameters, such as changes in body temperature, blood pressure, skin hydration, and concentration of triglyceride and albumin. Besides, it is also sensitive to environmental variation in temperature, humidity, atmospheric pressure, and carbon dioxide content. The mid-infrared spectroscopy technique which is based on the same physical principle as NIR, has strong limitation of poor penetration as light penetrates only a few micrometers inside the skin. The metabolic heat conformation has strong probability of interfering with the environmental conditions such as temperature and humidity.

For Raman spectroscopy, the scattering effects and the re-absorption of light in bio-tissues make the Raman signals hard to detect and require long spectral acquisition time. For Example, protein molecules produce a background fluorescence signal that is often equal to or larger than the Raman signal itself. For these reasons, the suitable detecting area on body for Raman measurement is the anterior chamber of the eye and aqueous humour. However, the above detecting areas are also sensitive parts on the body that only allows for applying a safe dose of incident irradiation power, and causes a poor detected result. The fluorescence-based techniques need to inject fluorescent chemicals into human body. The detected signal will degrade during detecting time. The optical spectroscopies techniques apply ultra-violet or visible light to perform measurement, but the wavelengths of ultra-violet and visible light are relatively short that cause high scattering. For bioimpedance measurement techniques, the glucose detecting signal will change with different detected individuals. Therefore, the bioimpedance measurement requires additional calibration for the base signal level in skin and underlying tissues among individuals. Beside, the bioimpedance measurement devices have multiple disadvantage and inconveniency for calibration. For example, the commercial product, Pendra, needs to changed the disposable detecting tape (Pendra tape) every 24 h. After once calibration, the same detecting area needs 1 hour for equilibrium that could be available for the next calibration, however the detecting results still have poor correlation of only 35%. For another commercial product of the bioimpedance measurement devices, Clarke EGA, the detecting results indicated 4.3% readings in error zone E, and the poor accuracy also shows in the post-marketing validation study. The patient using Clarke EGA must take rest for 1 hour for equilibration before the reading. The photo-acoustic technique needs strong pump power of light to perform the measurement that may cause potential damage to the tissue. For the currently microwave spectroscopy techniques, the currently applying wavelength of the electromagnetic wave has a poor spatial resolutions. Because the tissue has a high dielectric constant at this wavelength that leads to the electromagnetic wave hard to transmit trough the tissue, and unable to achieve a transmission measurement.

As described above, THz electromagnetic wave has potential to apply on noninvasive blood glucose detecting device. But up to the present, there is no available method or apparatus for measuring blood glucose concentration using absorption measurement of THz electromagnetic waves. Also, for the current non-invasive techniques, the glucose level is not monitored directly via blood, but via interstitial fluid, tissue, or body temperature that are correlated to blood glucose. Because the glucose level in tissue is easily to be affected, such as affected by metabolic activity of tissue, medication, blood pressure, body temperature etc., the currently techniques need a further complicated calibrations for a higher accuracy detecting result. To overcome and improve the disadvantage and inconveniency of current noninvasive blood glucose detecting technique, the present invention provides a noninvasive blood glucose novel detecting method. The blood glucose concentration can be correctly obtained without further calibration for physical parameters such as metabolic activity, medication, blood pressure or body temperature, and without a complicated calibration for obtaining a higher accuracy detecting result.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is an illustration of a blood glucose detecting device in the present invention.

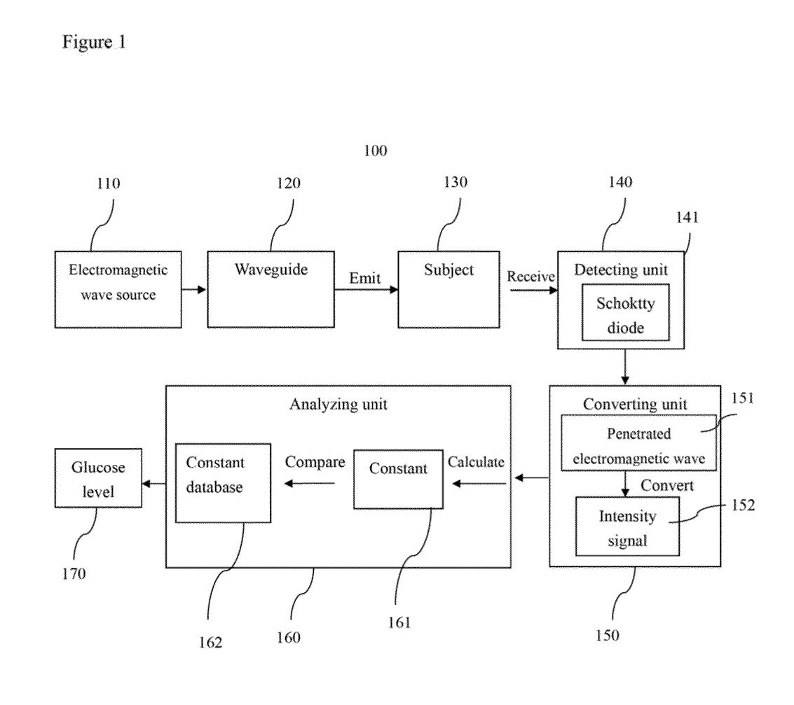

FIG. 2 is a related figure of the age and the corresponding blood glucose level of the KK-A^{y}/Ta Jcl mouse.

FIG. 3 shows the device described in example 2.

FIG. 4 is an illustration of applying the device in the present invention to screen a subject, wherein 210 is electromagnetic wave, 220 is a physical part of the subject and 221 is a blood vessel.

FIG. 5 is the electromagnetic wave absorption constant of a mouse ear.

FIG. 6 (a) is a photograph of a mouse ear and (b) is an electromagnetic wave absorption 2D image of the measured mouse ear.

FIG. 7 (a) is an electromagnetic wave absorption 2D image of a diabetes mouse ear (KK-A^{y}/Ta Jcl) in 4^{th} week old age and (b) is an electromagnetic wave absorption 2D image of the same mouse in the 5^{th} week old age.

FIG. 8 shows the device described in example 3.

FIGS. 9 (a)˜(d), respectively show the 2D electromagnetic wave absorption constant image of 4^{th}˜7^{th} week old control mouse ear.

FIGS. 10 (a)˜(d), respectively show the 2D electromagnetic wave absorption constant image of 4^{th}˜7^{th} week old diabetes mouse ear.

FIG. 11 shows a table of the weight, fasting glucose and glucose reaction in urine of the control and diabetes mouse.

FIG. 12 shows a blood glucose electromagnetic wave absorption constant regression curve.

FIG. 13 shows a device described in example 4.

FIG. 14 shows an electromagnetic wave absorption time domain signal.

FIG. 15 shows an electromagnetic wave absorption frequency domain signal.

FIG. 16 shows blood glucose electromagnetic wave absorption constants of patients, with a high and normal blood glucose level.

FIG. 17 shows a correlating figure of the electromagnetic wave absorption constant and the corresponding blood glucose level of human blood.

### SUMMARY OF THE INVENTION

The present invention provides a method for detecting a blood glucose level of a subject using an electromagnetic wave comprises the following steps: providing an electromagnetic wave using an electromagnetic wave source; emitting the electromagnetic wave to a subject, wherein the electromagnetic wave penetrates through the subject; using a detecting unit to receive and detect an intensity signal of the penetrated electromagnetic wave; calculating the intensity signal of the penetrated electromagnetic wave to obtain an electromagnetic wave absorption constant of the subject; comparing the electromagnetic wave absorption constant of the subject with data stored in a blood glucose electromagnetic wave absorption constant database; and obtaining a blood glucose level of the subject. The present invention also provides a device for detecting a blood glucose level of a subject using an electromagnetic wave comprises: an electromagnetic wave source for emitting an electromagnetic wave to the subject; a detecting unit for receiving and detecting the electromagnetic wave which penetrates through the subject; a converting unit for converting the penetrated electromagnetic wave into an intensity signal of the penetrated electromagnetic wave; and an analyzing unit for calculating the penetrated electromagnetic wave to obtain an electromagnetic wave absorption constant of the subject, and comparing the electromagnetic wave absorption constant of the subject with data stored in a blood glucose electromagnetic wave absorption constant database to obtain a blood glucose level of the subject.

### DETAILED DESCRIPTION OF THE INVENTION

In feature, a different blood glucose level has individual electromagnetic wave absorption ability. The present invention provides a novel blood glucose measuring device and method, using the high frequency electromagnetic wave, THz electromagnetic wave, measuring technology to quantify the blood glucose level and building a noninvasive method for blood glucose measurement to thereby replace the current invasive blood glucose detecting methods. The THz electromagnetic wave provided by present invention has a reasonable penetrating ability and safe operation power which overcomes the limitations and disadvantages of prior arts. In general, the present invention provides a fast, accurately, and continuously detecting method for daily glucose level monitoring.

The present invention relates to a method for detecting a blood glucose level of a subject using an electromagnetic wave, comprises the following steps:
(a) providing an electromagnetic wave using an electromagnetic wave source;
(b) emitting the electromagnetic wave to a subject, wherein the electromagnetic wave penetrates through the subject;
(c) using a detecting unit to receive and detect an intensity signal of the transmitted electromagnetic wave;
(d) calculating the intensity signal of the penetrated electromagnetic wave to obtain a electromagnetic wave absorption constant of the subject;
(e) comparing the electromagnetic wave absorption constant of the subject with data saved in a blood glucose electromagnetic wave absorption constant database; and
(f) obtaining a blood glucose level of the subject.

In one embodiment of the present invention, the electromagnetic wave source in step (a) the electromagnetic wave from the electromagnetic source is emitted to the subject via a waveguide unit, wherein the waveguide guides the electromagnetic wave parallel penetrate through the subject, which includes but not limited to a glass waveguide or a polyethylene (PE) waveguide.

The present invention is also related to a device 100 for detecting a blood glucose level of a subject 130 by an electromagnetic wave, comprises:

(a) an electromagnetic wave source 110 for emitting an electromagnetic wave to the subject 130;

(b) a detecting unit 140 for receiving and detecting the electromagnetic wave which penetrates through the subject 151;

(c) a converting unit 150 for converting the penetrated electromagnetic wave 151 into an intensity signal of the penetrated electromagnetic wave 152; and

(d) an analyzing unit 160 for calculating the penetrated electromagnetic wave to obtain an electromagnetic wave absorption constant 161 of the subject 130, and comparing the electromagnetic wave absorption constant of the subject with data saved in a blood glucose electromagnetic wave absorption constant database 162 to obtain a blood glucose level 170 of the subject 130.

The device in present invention further comprises a waveguide unit 120 for receiving the electromagnetic wave from the electromagnetic wave source 110 and transmitting the electromagnetic wave to the subject 130, wherein the waveguide guides the electromagnetic wave to parallel penetrate through the subject. In one embodiment a present invention, the electromagnetic wave source 110 includes but not limited to an aerial, microwave unit or millimeter wave unit. In one-embodiment, the detecting unit 140 further comprises a Schottky diode 141, wherein the Schottky diode is a room temperature-operated detecting component, allowing the present invention able to measure the blood glucose level of a subject under room-temperature.

In present invention, the intensity signal of the penetrated electromagnetic wave includes a 1D, 2D signal, wherein the 1D or 2D signal further includes electromagnetic wave intensity or a detecting location. In one preferred embodiment of the present invention, the 2D signal is a 2D image which is obtained by scanning a physical part of the subject together in 2D using the electromagnetic wave source and detecting unit. In other preferred embodiment, the 2D image is obtained by scanning a physical part of the subject in 2D only using the detecting unit.

In present invention, the device further includes a blood glucose electromagnetic wave absorption constant database 162, for saving the data of blood glucose levels and their correlated electromagnetic wave absorption constants.

The subject in present invention could be a living body, including a blood vessel(s) 221 passing through its physical part 220. In a preferred embodiment, the physical part 220 includes an ear(s), skin(s), finger(s), toe(s), lips or the skin linking between the fingers or toes. The electromagnetic wave 210 of the device can penetrate through the physical part 220 and the inside blood vessel 221 of the subject. In another preferred embodiment, the subject can further be fixed using a pair of films, wherein the films enable for the electromagnetic wave to penetrate, not absorb or reflect, through the subject.

The blood glucose electromagnetic wave absorption constant database in present invention provides a changing curve or a comparison table of blood glucose electromagnetic wave absorption constants. In one preferred embodiment, the changing curve or comparison table of blood glucose electromagnetic wave absorption constants is a regression function curve, and the formula of the regression function is:

y=a+bx (1)

where y is blood a glucose level and x is an electromagnetic wave absorption constant. Based on the above regression function, the data in the comparison table of blood glucose electromagnetic wave absorption constants can be further calculated. In the present invention, the data in the blood glucose electromagnetic wave absorption constant database is selected from the changing curve or the comparison table of blood glucose electromagnetic wave absorption constants.

The electromagnetic wave in the invention, generally, is a high frequency or a tetrahertz electromagnetic wave. The frequency range of the electromagnetic wave in the present invention is about 1 GHz˜10 THz, a preferable range is 10 GHz˜1 THz, more preferable, in a range of 50 GHz˜420 GHz.

The intensity signal of the penetrated electromagnetic wave can be converted by applying a Beer-Lamber Law formula:

$$Electromagnetic wave absorption constant ( α ) = \frac{ln ( {P}_{in} / {P}_{out} )}{t} , \qquad ( 2 )$$

wherein Pᵢₙ is a power (or intensity) of the emitted electromagnetic wave (background level), Pₒᵤₜ is a power (or intensity) of the penetrated electromagnetic wave and t is a thickness of the subject. Because different subjects may have different electromagnetic wave absorption ability, it may need to enter a parameter(s) for correcting the analyzing result of the present invention when calculating the electromagnetic wave absorption constant of different subjects. The parameter(s) described above includes but not limited to a thickness, skin or tissue electromagnetic wave absorption constant, or the blood glucose level and its corresponding electromagnetic wave absorption constant of the detected subject.

### EXAMPLES

The examples below are non-limiting and are merely representative of various aspects and features of the present invention.

### Example 1

### Animal Model

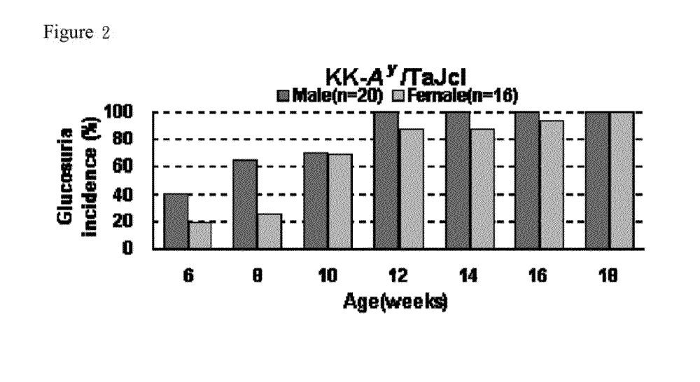

The present invention used 4˜7-week-old BALB/cByJNarl mouse (purchased from National Laboratory Animal Center in Taiwan) as a control model and used 4˜7-week-old KK-A^{y}/TaJcl mouse (purchased from CLEA Japan, Inc) as a type-2 diabetes model, which the blood glucose level raised during ageing, as shown in FIG. 2. The ear thickness of the mouse described above was 350 μm. The mouse was individually raised in a separated cage, and starved 8 hours before the experiment. Before experiment, the mouse was anesthetized using ketamine-xylazin (50 mg+15 mg/kg) by i.p. injection.

### Example 2

### Blood Glucose Detecting Device

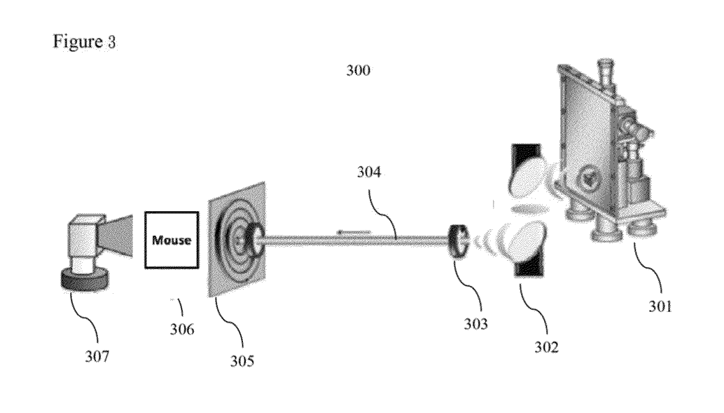

In this example, a device 300, as shown in FIG. 3, used a CW Gunn oscillator module 301 as a electromagnetic wave source, a parabolic mirror 302 for focusing and transmitting the electromagnetic wave from the source through a PE film 303 and to a waveguide unit 304. The waveguide unit 304 adopted a highly flexible THz sub-wavelength polyethylene (PE) fiber with a diameter of 240 μm, a length of 33 cm and a substantially low attenuation constant (5×10⁻³ cm⁻¹). To improve the spatial resolution, behind the fiber output end, a bull's-eye metallic spatial filter with a subwavelength aperture (diameter of 200 μm) 305 was intergraded to achieve both a high transmission power (10-fold higher than transmission through a single bare aperture of the same size) and near-field spatial resolution (240 μm<λ/4) beyond the diffraction limit.

The measurement frequency range of electromagnetic wave in this example was about 320˜420 GHz. 340 GHz was selected as a working frequency. The electromagnetic wave power on the surface of the mouse ear was about 1 mW. A room-temperature-operated Schoktty diode detector 307 (Virginia Diode, Inc, model WR-2.8, response time <5 μsec.) was used for receiving a transmitted electromagnetic wave 306. The detecting area was 10 mm×10 mm and the imaging time is 3 mins/100×100 pixels. The preferred working condition of the present device was under room temperature 23° C. and with about 50% humidity.

Operation of a Blood Glucose Detecting Device

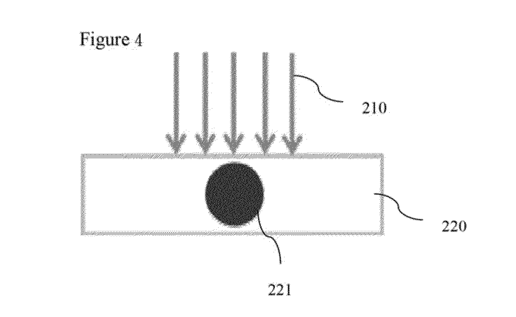

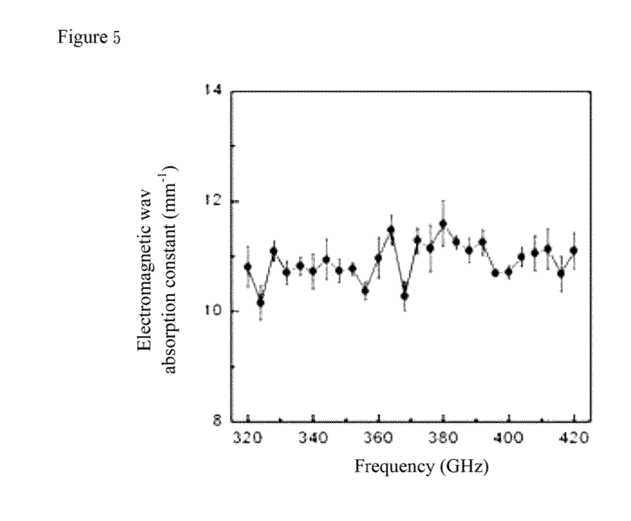

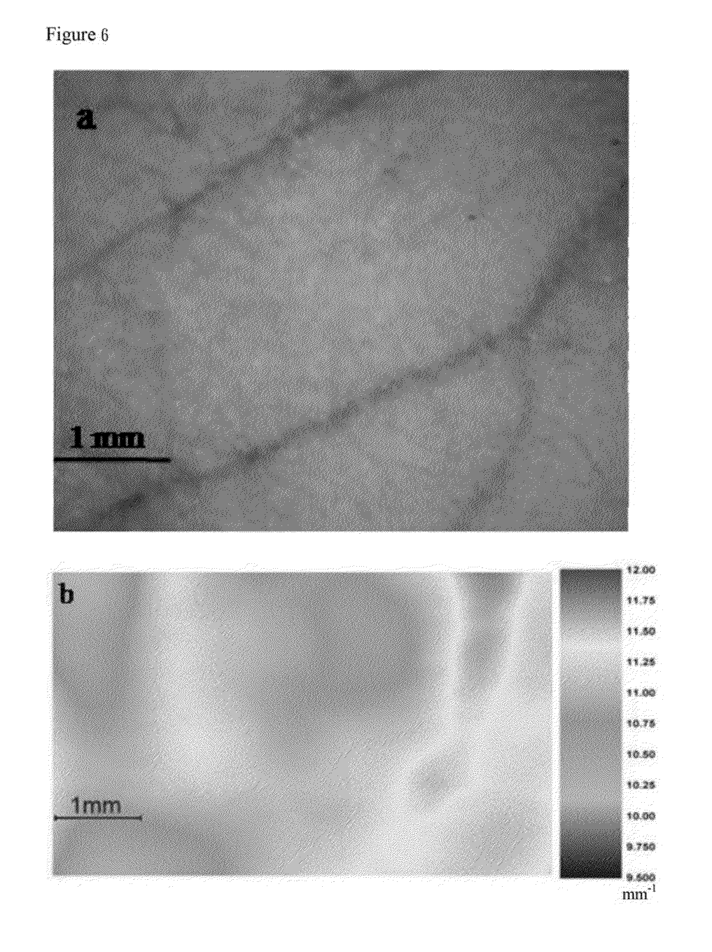

First, without putting any subject on the device, the present example used the component of electromagnetic wave source (waveguide and bull's-eye metallic spatial filter with a sub wavelength aperture) to scan in 2D an area of 6 mm×4 mm in air and obtained a background electromagnetic wave absorption constant (or 2D image). The detected images of the following experiment in this example were normalized to the background for correcting the angle-dependent bending loss. After above steps, the mouse were anesthetized and moved to the detecting area of the present device. The mouse ear was sandwiched by two acrylic films and fixed by a 6 mm×4 mm metallic aperture. The distance between the bottom of the mouse ear and the surface of the metallic bull's-eye structure was about 250 μm. The device scanned the mouse ear using the bull's-eye metallic spatial filter with a subwavelength aperture 305 and the detecting unit 307 by a 2D scanning method (FIG. 4), and obtained an electromagnetic wave intensity 2D image. The 2D image and the background level were then calculated using the Beer-Lamber Law to obtain a 2D electromagnetic wave absorption constant image, as shown in FIG. 6(b). FIG. 5 showed the screening results of mouse ear with crossed blood vessels by applying the present device using a serial electromagnetic wave frequency. (FIG. 5 is not a background image, but a screening result of a living body.)

FIG. 6, including (a) and (b), was a photograph and its corresponding 2D electromagnetic wave absorption constant image of the measured mouse ear. In FIG. 6(b), in the mouse ear, the area with a passing blood vessel had a higher electromagnetic wave absorption constant compared to the surrounding tissue (α=10.5 mm⁻¹˜11.2 mm⁻¹). Besides, the subject with higher blood glucose level had higher electromagnetic wave absorption constant. Therefore, the 2D electromagnetic wave absorption constant image could be used to recognize the position of the capillary and the surrounding tissue in the ear.

Detecting Results of the Diabetes Animal Model

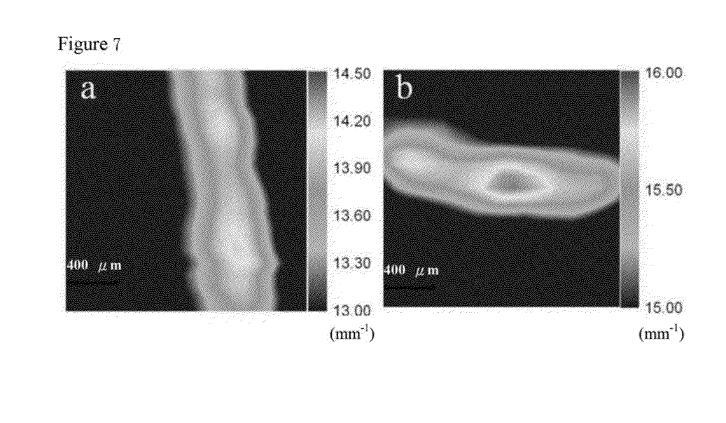

The present example used 4-week-old KK-A^{y}/TaJcl mouse as a type-II diabetes model. The detected 2 mm×2 mm electromagnetic wave absorption image of the same mouse with 4 and 5 weeks age were showed in FIGS. 7(a) and (b). The blood glucose electromagnetic wave absorption constant was found to be increased from 14.5 mm⁻¹ to 16 mm⁻¹. It was also noticed that the absorption constant of the surrounding tissues was also increased, which suggested that the glucose content in the surrounding tissues was also increased.

### Example 3

### Blood Glucose Detecting Device

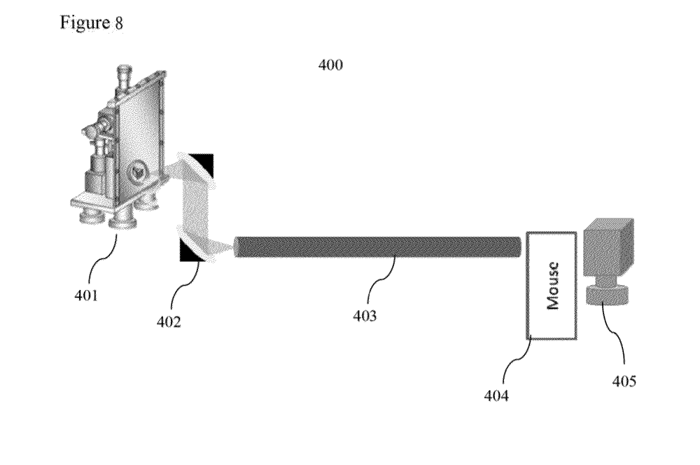

In the present example, a device 400 (FIG. 8) used a CW Gunn oscillator module 401 as a electromagnetic wave source, and a parabolic mirror 402 for focusing and transmitting the electromagnetic wave from the source to a waveguide unit 403. The waveguide unit adopted a THz glass waveguide with an inner-diameter of 9 mm, an outer-diameter of 13 mm, a thickness of 2 mm and a length of 30 cm, wherein the glass waveguide had a substantially low attenuation constant (2×10⁻² cm⁻¹ @340 GHz). To improve the spatial resolution, a 300 um×700 um aperture was set in front of a Schoktty diode in a detecting unit 405.

The measurement frequency range of the electromagnetic wave in this example was about 320˜420 GHz. 340 GHz was selected as a working frequency. The electromagnetic wave power on the surface of a subject 404, such as a mouse, was about 1 mW. A room-temperature-operated Schottky diode detector (Virginia Diode, Inc, model WR-2.8, response time <5 μsec.) was used for receiving the penetrated electromagnetic wave 306. The detecting area was 10 mm×10 mm and the imaging time is (3 minutes)/100×100 pixels. The preferred working condition of the present device was under room temperature 23° C. and with about 50% humidity.

Operation of Blood Glucose Detecting Device

First, without putting any subject on the device, the present example use the component of the electromagnetic wave source and detecting unit to scan in 2D an area of 10 mm×10 mm in air and obtained a background electromagnetic wave absorption constant (or 2D image). After above steps, the mouse was anesthetized and moved to the detecting area of the present device. The mouse ear was sandwiched by two acrylic apertures with a diameter of 9 mm. The distance between the mouse ear and the surface of the detecting unit is about 2 mm. The device scanned the mouse ear using the detecting unit 405 by a 2D scanning method, and obtained an electromagnetic wave 2D intensity image. The 2D image and background level were calculated using Beer-Lamber Law to obtain a 2D electromagnetic wave absorption constant image.

The detecting results of the presenting device on different physical parts of mouse (at 340 GHz) were: 11 mm⁻¹ of skin, 10 mm⁻¹ of tissue, 11 mm⁻¹ of red blood cell, 11 mm⁻¹ of white blood cell and 12 mm⁻¹ of water. Besides, due to the resolution size (300 μm) was 2 times greater than the size of the capillary, the present invention would not be limited by the blood vessel size of the subject.

Detecting Results of the Control and Diabetes Animal Model

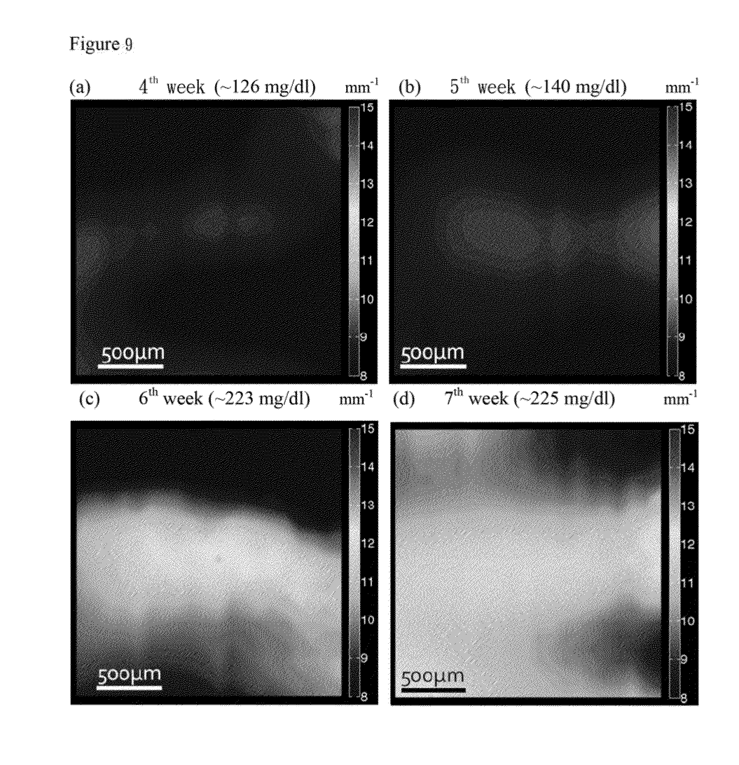

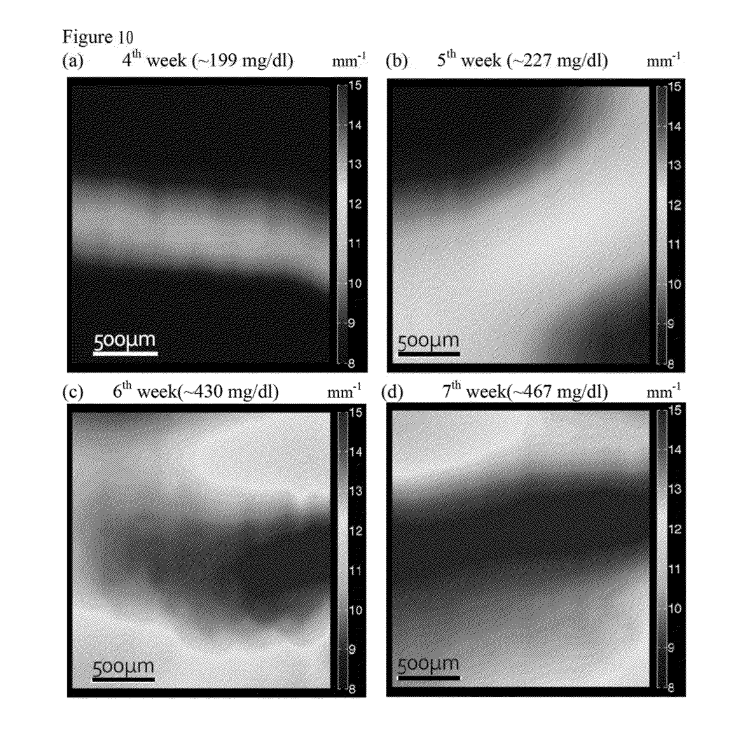

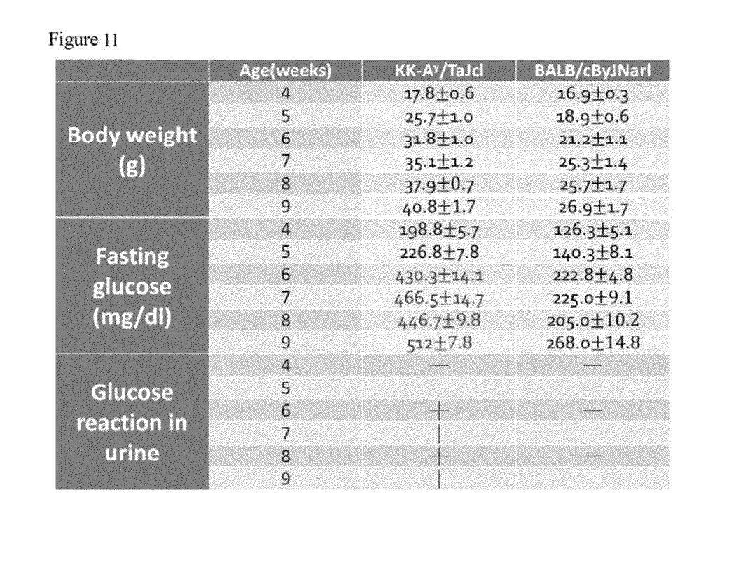

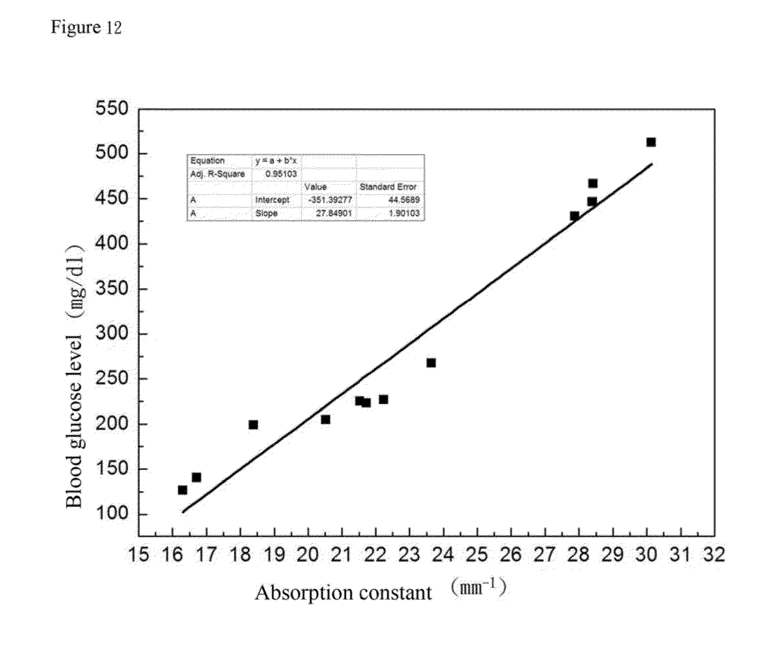

The present example detected the electromagnetic wave absorption constant of the 4-7-week-old normal and diabetes mouse. The 2D electromagnetic wave absorption constant image of normal mouse showed in FIGS. 9 (a)˜(d), and diabetes mouse showed in FIGS. 10 (a)˜(d). FIG. 11 showed the weight, the fasting glucose and glucose reaction in urine of the control and diabetes mouse. After calculated the above data into a regression curve (FIG. 12), the variation of the regression curve was found identically to the mouse blood glucose level. Therefore, the physical blood glucose level could be obtained using the regression curve with electromagnetic wave absorption constant detected by the present invention.

### Example 4

### Blood Glucose Detecting Device

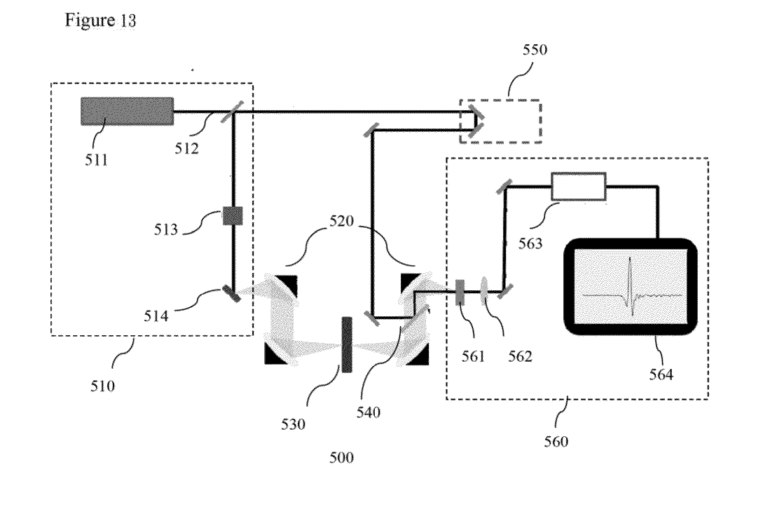

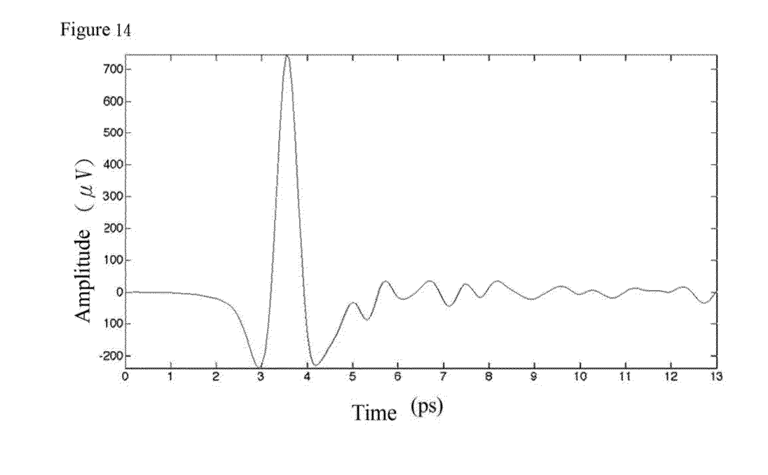

In the present example, the device 500 used terahertz time-domain spectroscopy (THz-TDS, FIG. 13). An electromagnetic wave source 510 used a Ti:Sapphire femtosecond laser 511 to emit a pulse laser to a beam splitter 512. The pulse laser was separated by the beam splitter into a pump beam and a probe beam. The pump beam was transmitted to an acousto-optic modulator (AOM) 513 and then focused onto a p-type InAs crystal 514 which converted the pump beam into a THz electromagnetic wave. The THz electromagnetic wave was focused and transmitted to a measured subject or sample (blood sample in this example) 530 by a parabolic mirror 520. Because a different blood glucose level is accompanied by a different electromagnetic absorption coefficient, a different subject would have different response intensity to the electromagnetic wave which penetrated through the subject. Using a parabolic mirror, the penetrated electromagnetic wave was transmitted and co-introduced with the probe beam through a silicon wafer 560 into a ZnTe crystal 561 which converted the penetrated electromagnetic wave into an optical wave. The optical wave and the probe beam produced a nonlinear effect. The delayed optical path of the probe beam could be obtained by a micro-control translation stage 550, and used for measuring the penetrated electromagnetic wave signal in any time point to performed different intensity change. All optical waves finally were transmitted through a convex 562, and the signal intensity of those optical waves were detected by a balance detector 563 (Zomega—ABL100 auto-balance detector). The detected signals were transmitted to a signal receptor 564 (Stanford research—SR844 Lock-in amplifier) to obtain a THz electromagnetic wave time domain signal (FIG. 14).

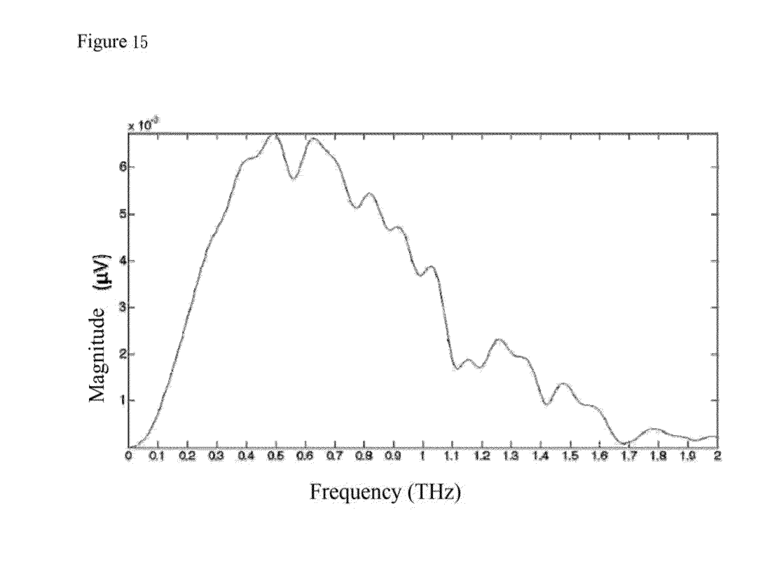

The electromagnetic wave absorption frequency domain signal (FIG. 15) was obtained by further calculating the information on THz electromagnetic wave time domain signal by Fast Fourier Transform (formula 3):

$${X}_{k} = {\sum }_{n = 0}^{N - 1} {x}_{n} {e}^{- i2π k \frac{n}{N}} , \\ k = 0 , 1 , … , N - 1. \qquad ( 3 ) ,$$

wherein xₙ was a time domain signal, Xₖ was a frequency domain signal and N was signal number (1024 in this example).

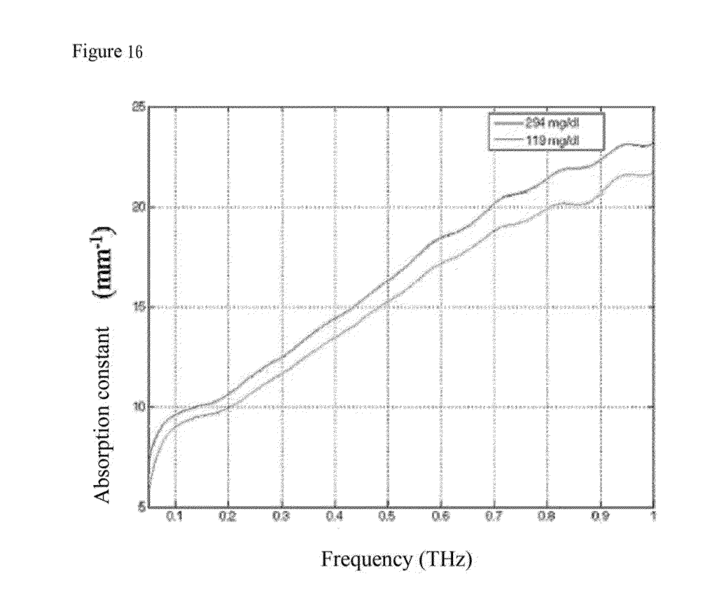

The present example further calculated the electromagnetic wave absorption frequency domain signal using the Beer Lambert Law formula and obtained as an electromagnetic wave absorption constant signal as showed in FIG. 16. The results were summarized as below: the blood sample with a higher glucose level had better electromagnetic wave absorption ability than a lower one. Besides, the absorption constant of samples increased with the frequency of the detecting electromagnetic wave.

Detecting Results of the Human Blood Sample

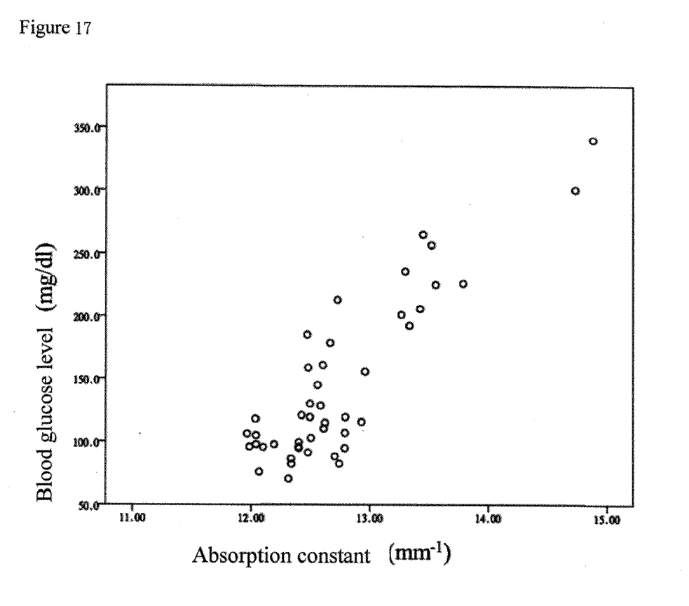

The present example detected 50 patient blood samples. FIG. 17 showed that the electromagnetic wave absorption constants and the blood glucose levels could perform a linear relationship (at 0.34 THz) with a correlation coefficient of 0.872 (p<0.0001).

While the invention has been described and exemplified in sufficient detail for those skilled in this art to make and use it, various alternatives, modifications, and improvements should be apparent without departing from the spirit and scope of the invention.

One skilled in the art readily appreciates that the present invention is well adapted to carry out the objects and obtain the ends and advantages mentioned, as well as those inherent therein. The animals, processes and methods for producing them are representative of preferred embodiments, are exemplary, and are not intended as limitations on the scope of the invention. Modifications therein and other uses will occur to those skilled in the art. These modifications are encompassed within the spirit of the invention and are defined by the scope of the claims.

What is claimed is:

1. A method for detecting a blood glucose level of a subject using an electromagnetic wave comprises the following steps:
(a) providing an electromagnetic wave using an electromagnetic wave source;
(b) emitting the electromagnetic wave to a subject, wherein the electromagnetic wave penetrates through the subject;
(c) using a detecting unit to receive and detect an intensity signal of the penetrated electromagnetic wave;
(d) calculating the intensity signal of the penetrated electromagnetic wave to obtain an electromagnetic wave absorption constant of the subject;
(e) comparing the electromagnetic wave absorption constant of the subject with data stored in a blood glucose electromagnetic wave absorption constant database; and
(f) obtaining a blood glucose level of the subject.

2. The method of claim 1, wherein in step (b) the electromagnetic wave is emitted to the subject via a waveguide unit.

3. A device for detecting a blood glucose level of a subject using an electromagnetic wave comprises:
(a) an electromagnetic wave source for emitting an electromagnetic wave to the subject;
(b) a detecting unit for receiving and detecting the electromagnetic, wave which penetrates through the subject;
(c) a converting unit for converting the penetrated electromagnetic wave into an intensity signal of the penetrated electromagnetic wave; and
(d) an analyzing unit for calculating the penetrated electromagnetic wave to obtain an electromagnetic wave absorption constant of the subject, and comparing the electromagnetic wave absorption constant of the subject with data stored in a blood glucose electromagnetic wave absorption constant database to obtain a blood glucose level of the subject.

4. The device of claim 3, further comprising a waveguide unit for receiving the electromagnetic wave from the electromagnetic wave source and transmitting the electromagnetic wave to the subject.

5. The device of claim 3, wherein the intensity signal of the penetrated electromagnetic wave comprises a 1D signal, 2D image.

6. The device of claim 5, wherein the 1D signal, 2D image includes electromagnetic wave intensity or a detecting location.

7. The device of claim 3, wherein the analyzing unit calculates the penetrated electromagnetic wave using a Beer-Lamber Law formula to obtain an electromagnetic wave absorption constant of the subject.

8. The device of claim 3, wherein the subject is a living body, including a blood vessel(s) passing through its physical part.

9. The device of claim 8, wherein the physical part comprising an ear(s), skin(s), finger(s), toe(s) lips, or the skin linking between the fingers or toes.

10. The device of claim 3 further comprises a blood glucose electromagnetic wave absorption constant database for storing data of blood glucose electromagnetic wave absorption constants.

11. The device of claim 10, wherein the database is a changing curve or a comparison table of blood glucose electromagnetic wave absorption constants.

12. The device of claim 3, wherein the electromagnetic wave is a high frequency electromagnetic wave and has a frequency range of 1 GHz˜10 THz.

13. The device of claim 12, wherein the frequency range of the electromagnetic wave is preferable in a range of 10 GHz˜1 THz.

14. The device of claim 13, wherein the frequency range of the electromagnetic wave is more preferable in a range of 50 GHz˜420 GHz.
